(19) United States
(12) Patent Application Publication
(10) Pub. No.: US 2025/0375360 A1
(43) Pub. Date: Dec. 11, 2025

(54) COSMETIC COMPOSITION COMPRISING THREE DIFFERENT FORMULATIONS

(71) Applicant: AMOREPACIFIC CORPORATION, Seoul (KR)

(72) Inventors: Choonbok JEONG, Seoul (KR); Seung Hwan YI, Seoul (KR)

(73) Assignee: AMOREPACFIC CORPORATION, Seoul (KR)

(21) Appl. No.: 19/223,800

(22) Filed: May 30, 2025

(30) Foreign Application Priority Data
Jun. 10, 2024 (KR) 10-2024-0075107

(51) Int. Cl. A61K 8/04; A61K 8/06; A61Q 19/00

(52) U.S. Cl. CPC A61K 8/042; A61K 8/062; A61K 8/064; A61Q 19/00; A61K 2800/882

(57) ABSTRACT

A cosmetic composition including three different chemical formulations, wherein the first phase includes an oil-in-water (O/W) type or an aqueous dispersion, the second phase includes a silicone gel and a water gel, and the third phase includes a water-in-silicone (W/S) type or a water-in-oil (W/O) type.

### CROSS-REFERENCE TO RELATED APPLICATION

This application claims priority to and the benefit of Korean Patent Application No. 10-2024-0075107 filed in the Korean Intellectual Property Office on Jun. 10, 2024, the entire contents of which are incorporated herein by reference.

### BACKGROUND OF THE INVENTION

### (a) Field of the Invention

The present disclosure relates to a cosmetic composition including three different chemical formulations.

### (b) Description of the Related Art

Typically, cosmetics may be classified into skincare products such as toners, lotions, and creams that may moisturize the skin and makeup products such as makeup bases, foundations, and the like. Recently, interests in hybrid makeup cosmetics having makeup functions and simultaneously, skin care functions such as skin nutrition, skin moisturizing, skin barrier reinforcement, and the like are increasing. Examples of the hybrid products include foundations containing UV protection ingredients, lipsticks with moisturizing ingredients, etc.

On the other hand, a cosmetic composition may have a chemical formulation such as an oil-in-water (O/W) type, a water-in-oil (W/O) type, a silicone-in-water (W/S) type, or the like. The oil-in-water chemical formulation is characterized to feel relatively light on the skin and may be easily washed away by sweat or water, while the water-in-oil chemical formulation may be less washed away by water and is characterized to feel heavy and sticky on the skin. In addition, the silicone-in-water chemical formulation a silky texture and relatively refreshing feeing of use and also, characteristics of improving skin texture and protecting the skin.

### SUMMARY OF THE INVENTION

An embodiment provides a cosmetic composition that is uniformly mixed even when three different chemical formulations are included, has a light feeling on the skin, and has excellent adhesion and durability.

According to an embodiment, a cosmetic composition includes three different chemical formulations, wherein the first phase includes an oil-in-water (O/W) type or an aqueous dispersion, the second phase includes a silicone gel and a water gel, and the third phase includes a water-in-silicone (W/S) type or a water-in-oil (W/O) type.

The first phase may include polyacrylate crosspolymer-6, sodium polyacryloyldimethyltaurate, an ammonium acryloyldimethyltaurate/VP copolymer, or a combination thereof.

The second phase may include 35 to 90 wt % of silicone gel based on a total weight of the second phase.

The second phase may be a silicone-in-water (W/S) type including an oil-phase component, and the oil-phase component may be included in an amount of 60 wt % to 90 wt % based on a total weight of the second phase.

The water gel may include gellan gum, xanthan gum, galactomannan, glucomannan, guar gum, locust bean gum, pluronic, agar, carrageenan, or a combination thereof.

The third phase may include a volatile oil selected from isooctane, isodecane, isododecane, isohexadecane, isoparaffin, dimethylpolysiloxane, cyclomethicone, cyclotetrasiloxane, cyclopentasiloxanecyclohexasiloxane, phenyl trimethicone, methyl trimethicone, dimethicone, trisiloxane, or a combination thereof.

The third phase may include a volatile oil, and the volatile oil may be included in an amount of 25 wt % or less based on a total weight of the cosmetic composition.

The cosmetic composition may include 10 to 60 wt % of the first phase, 10 to 60 wt % of the second phase, and 10 to 60 wt % of the third phase, based on a total weight of the composition.

The cosmetic composition may include 100 to 150 parts by weight of the first phase and the third phase respectively, based on 100 parts by weight of the second phase.

Viscosities of the first to third phases may each be 15,000 cps or less.

The first phase, the second phase, and the third phase in the cosmetic composition may not be mixed with each other, but may be mixed immediately before use.

The cosmetic composition may be such that the second phase may be located between the first phase and the third phase prior to use.

Another embodiment provides a cosmetic including the aforementioned cosmetic composition in a multi-tube container.

The cosmetic may be used as one or more of a makeup primer, a makeup base, a foundation, a concealer, a pact, a powder pact, a lipstick, a lip gloss, a powder, an eyeliner, an eyebrow pencil, an eye shadow, a blusher, a sunscreen, a lotion, a cream, an essence, a cleansing oil, and a makeup remover.

A cosmetic composition including three different chemical formulations according to an embodiment of the present invention may exhibit excellent adhesion and durability while feeling light on the skin due to the uniform mixing of the three different chemical formulations. Additionally, it has both skin care and makeup functions, and thus it can simplify the makeup process.

### BRIEF DESCRIPTION OF THE DRAWINGS

FIG. 1A is a photograph showing the appearance of the composition according to Comparative Example 1 immediately after being discharged from the container.

FIG. 1B is a photograph showing the appearance of a composition mixed according to Comparative Example 1.

FIG. 2A is a photograph showing the appearance of the composition according to Example 1 immediately after being discharged from the container.

FIG. 2B is a photograph showing the appearance of a composition mixed according to Example 1.

FIG. 3 is a photograph showing the appearance during mixing of a composition according to Example.

FIG. 4 is a photograph showing the appearance during mixing of the composition according to Comparative Example 2.

FIG. 5 is a photograph showing the appearance during mixing of the composition according to Comparative Example 3.

### DETAILED DESCRIPTION OF THE EMBODIMENTS

Hereinafter, example embodiments of the present invention will be described in detail. However, these example embodiments are only examples and do not limit the present invention. However, this disclosure may be embodied in many different forms and is not construed as limited to the example embodiments set forth herein.

A cosmetic composition including three different chemical formulations according to an embodiment includes a first phase including an oil-in-water (O/W) type or an aqueous dispersion, a second phase including a silicone gel and a water gel, and a third phase including a water-in-silicone (W/S) type or a water-in-oil (W/O) type.

Even though the cosmetic composition includes ingredients of three different chemical formulations, because the chemical formulations may be uniformly mixed one another, each of the chemical formulations may exhibit its own function at maximum. Accordingly, the cosmetic composition may not only have light feeling of use on the skin but also effectively adheres to the skin and in addition, allow makeup to last for a long time.

The cosmetic composition, which includes three different chemical formulations, may supply moisture to the skin and simultaneously refine skin texture and improve skin tone. Accordingly, the cosmetic composition alone may cover everything from skincare to makeup, which may eliminate the use of various products, shorten makeup time, and the like to simplify makeup and thereby, provide convenience.

In addition, if conventional products made of each of the first to third phase chemical formulations are separately used, it is difficult to appropriately control an amount of each chemical formulation. For example, if any one of the chemical formulations is excessively used, the skin may be provided with excessive moisture according to the excessively used chemical formulation, which may cause makeup to pile up or skin tone-up to be too much or too little. However, the cosmetic composition, in which three different chemical formulations are mixed and used in a predetermined amount for each use, is easy to control the amount and in addition, provides light feel of use on the skin and simultaneously, minimize makeup from caking or cracking for each use.

In an embodiment, the cosmetic composition includes the first phase and thus exhibits skin care effects such as moisture supply to the skin and the like. The first phase may include an oil-in-water (O/W) or aqueous dispersion chemical formulation. The oil-in-water chemical formulation has an aqueous phase as a continuous phase and a dispersed phase as an oil phase, but the aqueous dispersion chemical formulation is an aqueous phase chemical formulation including no oil phase.

The first phase may include polyacrylate cross polymer-6, sodium polyacryloyldimethyl taurate, ammonium acryloyldimethyltaurate/VP copolymer, a polyol, purified water, or a combination thereof, but is not limited thereto.

The polyol refers to a compound including plurality of hydroxy groups, and is, for example, glycerin, butylene glycol, or a combination thereof, but is not limited thereto.

In an embodiment, the first phase may be included in an amount of 10 wt % to 60 wt %, for example, 10 wt % to 60 wt %, 15 wt % to 60 wt %, 20 wt % to 60 wt %, 25 wt % to 60 wt %, 30 wt % to 60 wt %, 10 wt % to 55 wt %, 10 wt % to 50 wt %, 10 wt % to 45 wt %, 10 wt % to 40 wt %, 20 wt % to 50 wt % based on the total weight of the cosmetic composition, but is not limited thereto. When the first phase is included in the above amount, the skin moisturizing effect of the cosmetic composition can be maximized.

In an embodiment, the cosmetic composition can fill in unevenness caused by pores, wrinkles, etc. of the skin and even out skin texture by including a second phase, thereby increasing the adhesion of the cosmetic composition to the skin.

In an embodiment, the second phase may include a silicone gel and a water gel. The second phase may uniformly mix various chemical formulations in the cosmetic composition including both silicone gel and water gel, and can improve the feeling of use of the cosmetic composition.

For example, the silicone gel may be easily applied using commercially available raw materials such as KSG-15. 16. 18. 41, 42, 43, 44, 210, 310, 320, 330, 340, 710, 830, 840 from Shinestu, Dow Corning EL-8051IN Silicone Organic Elastomer Blend, Dow Corning EL-8052IH Silicone Organic Elastomer Blend, Dow Corning EL-8050ID Silicone Organic Elastomer Blend from Dow Corning, but silicone gel raw materials specially manufactured according to the needs of those skilled in the art may also be applied, and is not limited thereto.

In an embodiment, the silicone gel may be included in an amount of 35 wt % to 90 wt %, for example, 40 wt % to 90 wt %, 35 wt % to 85 wt %, 40 wt % to 85 wt %, 35 wt % to 80 wt %, 40 wt % to 80 wt %, 40 wt % to 75 wt %, 40 wt % to 70 wt % based on a total weight of the second phase, but is not limited thereto.

For example, the water gel may be gellan gum, xanthan gum, galactomannan, glucomannan, guar gum, locust bean gum, pluronic, agar, carrageenan, biosaccharide gum, cellulose, sclerotium gum, agarose, or a combination thereof, for example, gellan gum, xanthan gum, galactomannan, glucomannan, guar gum, locust bean gum, pluronic, agar, carrageenan, or a combination thereof, but is not limited thereto. Additionally, the water gel may further include glycerin, butylene glycol, propanediol, methylpropanediol, dipropylene glycol, hexanediol, purified water, or a combination thereof, but is not limited thereto.

In an embodiment, the water gel may include 10 wt % to 50 wt %, for example, 10 wt % to 45 wt %, 10 wt % to 40 wt %, 10 wt % to 35 wt %, 10 wt % to 30 wt %, 15 wt % to 50 wt %, 15 wt % to 45 wt %, or 15 wt % to 40 wt % based on a total weight of the second phase, but is not limited thereto.

In an embodiment, the second phase may be a silicone-in-water type, and may be, but is not limited to, a silicone-in-water type in which an aqueous phase is dispersed in a continuous phase including an oil phase including a silicone gel. The oil-phase component may be included in an amount of 60 wt % to 90 wt %, for example, 65 wt % to 90 wt %, 70 wt % to 90 wt %, 60 wt % to 85 wt %, 60 wt % to 80 wt % based on a total weight of the second phase, but is not limited thereto.

In an embodiment, the second phase may further include an emulsifier to stabilize the chemical formulation. For example, the emulsifier may be, but is not limited to, Laurylpolyglyceryl-3 polydimethylsiloxy ethyl dimethicone.

The emulsifier may not be used, and may be used in small amounts. The emulsifier may be included in an amount of 0 wt % to 5 wt %, for example, 0 wt % to 4 wt %, 0 wt % to 3 wt %, 0 wt % to 2 wt %, 0 wt % to 1 wt %, 0.01 wt % to 5 wt %, 0.1 wt % to 5 wt %, 0.1 wt % to 4 wt %, or 0.1 wt % to 3 wt % based on a total weight of the second phase, and desirably, does not include an emulsifier, but is not limited thereto.

In an embodiment, the second phase may be included in an amount of 10 wt % to 60 wt %, for example, 10 wt % to 60 wt %, 15 wt % to 60 wt %, 20 wt % to 60 wt %, 25 wt % to 60 wt %, 30 wt % to 60 wt %, 10 wt % to 55 wt %, 10 wt % to 50 wt %, 10 wt % to 45 wt %, 10 wt % to 40 wt %, or 20 wt % to 50 wt % based on a total weight of the cosmetic composition, but is not limited thereto. When the above second phase is included in the above amount, the cosmetic composition can maximize the effect of evenly improving skin texture.

In an embodiment, the cosmetic composition can cover blemishes, freckles, wrinkles, pores, etc. of the skin by including a third phase to even out skin color.

The third phase may include a water-in-silicone (W/S) type or a water-in-oil (W/O) type. The silicone-in-water type refers to a chemical formulation in which the continuous phase includes silicone oil and the dispersed phase includes an aqueous phase, and the water-in-oil type refers to a chemical formulation in which the continuous phase includes an oil phase and the dispersed phase includes an aqueous phase.

In an embodiment, the oil phase of the third phase is a material that does not mix well with the water, and may be an oil, an organic solvent or a mixture thereof that is liquid at room temperature and atmospheric pressure, and may include, for example, a volatile oil or a non-volatile oil, and may include, for example, silicone oil, but is not limited thereto. The non-volatile oil may refer to oil having a residual oil content of 80% or more when stored in a constant temperature bath at 55° C. for 1 hour.

In an embodiment, the volatile oil may be, for example, a hydrocarbon-based volatile oil or a silicone-based volatile oil. Examples of the hydrocarbon-based volatile oil may include, but are not limited to, isooctane, isodecane, isododecane, isohexadecane, isoparaffin, or a combination thereof. Additionally, the silicone-based volatile oil may be, for example, dimethylpolysiloxane, cyclomethicone, cyclotetrasiloxane, cyclopentasiloxane, cyclohexasiloxane, phenyl trimethicone, methyl trimethicone, dimethicone, trisiloxane, or a combination thereof.

The volatile oil may be, but are not limited to, dimethylpolysiloxane, cyclomethicone, cyclotetrasiloxane, cyclopentasiloxane, cyclohexasiloxane, phenyltrimethicone, methyltrimethicone, dimethicone, trisiloxane, or a combination thereof.

In an embodiment, the volatile oil may be included in an amount of 25 wt % or less, for example, 23 wt % or less, 22 wt % or less, 21 wt % or less, 20 wt % or less, 19 wt % or less, 18 wt % or less, 17 wt % or less, 16 wt % or less, or 15 wt % or less based on a total weight of the cosmetic composition, but is not limited thereto. When the above volatile oil is included in the above content range, the third phase has a similar volatilization rate as the first and second phases, so that the three chemical formulations are more evenly mixed, and skin irritation or dryness can be minimized when the cosmetic composition is used.

In an embodiment, the third phase may be included in an amount of 10 wt % to 60 wt %, for example, 15 wt % to 60 wt %, 20 wt % to 60 wt %, 10 wt % to 55 wt %, 10 wt % to 50 wt %, 10 wt % to 45 wt %, 10 wt % to 40 wt %, or 20 wt % to 50 wt % based on a total weight of the cosmetic composition, but is not limited thereto. When the third phase is included in the above amount, the cosmetic composition can maximize the skin tone improvement effect.

In an embodiment, the cosmetic composition may include 100 parts by weight to 150 parts by weight, for example, 100 parts by weight to 140 parts by weight, or 100 parts by weight to 130 parts by weight, of the first phase based on 100 parts by weight of the second phase, but is not limited thereto. In addition, the third phase may be included in an amount of 100 to 150 parts by weight, for example, 100 to 140 parts by weight, or 100 to 130 parts by weight, based on 100 parts by weight of the second phase, but is not limited thereto. For example, the composition may include, but is not limited to, similar amounts of the first phase, the second phase, and the third phase, respectively. By including the first to third phases in the above content ratio, the uniform mixing of the three chemical formulations can be maximized, and the skin moisture replenishment, skin texture improvement, and skin tone-up improvement effects of the composition can be maximized.

In an embodiment, the first to third phases may each have a viscosity of 15,000 cps or less, for example, 14,000 cps or less, 13,000 cps or less, 12,000 cps or less, 11,000 cps or less, or 10,000 cps or less, but are not limited thereto. When the first to third phases have the above viscosity, the three chemical formulations can be easily mixed, and the cosmetic composition including them can be discharged from the container with little force.

In an embodiment, the first to third phases each have a viscosity within the above range and a viscosity difference between each chemical formulation is 2,000 cps or less, for example, 1,900 cps or less, 1,800 cps or less, 1,700 cps or less, 1,600 cps or less, or 1,500 cps or less, but is not limited thereto. For example, a deviation of the viscosity of the first and second phases is 2,000 cps or less, a deviation of the viscosity of the second and third phases is 2,000 cps or less, and a deviation of the viscosity of the first and third phases is 2,000 cps or less. When the viscosity deviations of the first to third phases are within the above range, the three chemical formulations can be easily mixed, and the cosmetic composition containing them can be discharged from the container with little force.

In an embodiment, the cosmetic composition may have the first phase, the second phase, and the third phase not mixed with each other, but mixed immediately before use. Specifically, the three chemical formulations included in the cosmetic composition are separated and not mixed, and when used, the chemical formulations of the first to third phases are discharged simultaneously and mixed for use.

In an embodiment, the cosmetic composition may be such that the second phase is located between the first phase and the third phase prior to use, and the second phase is located between the first phase and the third phase immediately prior to use, so that the three chemical formulations of the cosmetic composition may be more uniformly mixed.

Another embodiment provides a cosmetic including the cosmetic composition in a multi-tube container. The cosmetic composition may be stored in a multi-tube container so that the three chemical formulations within the composition may be stored separately and mixed immediately after being discharged from the container when used. Therefore, when stored, because one chemical formulation with relatively higher adsorption is not mixed with the other formulations, each of the chemical formulations may be stored without deformation and maintain its own original functions, and when used, each of the chemical formulations may stably exhibit the functions. In addition, because an amount of each chemical formulation may be appropriately adjusted, when the cosmetic composition is used, light feeling of use on the skin may be secured, and simultaneously, skin adhesion and durability of the cosmetic composition may be increased.

For example, the multi-tube container may use any container capable of separately storing the three chemical formulations without limitations in shape and size, for example, a pack, a jar, an airless container, a pump container, a vacuum container, a blow container, a tube, a bottle, or a combination thereof, for example, an airless container, a pump container, or a vacuum container but is not limited thereto.

For example, the multi-tube container may include a multi-tube container having three tubes, so that the chemical formulations contained in each tube may not be mixed, for example, a multi-tube pump container or a multi-tube airless container but is not limited thereto. The three tubes are arranged closely side by side or arranged to have a circular horizontal cross-section. For example, three tubes are arranged closely side by side, so that the horizontal cross-section of the three tubes may be a combination of three rectangular shapes, but is not limited thereto.

For example, in the multi-tube container, a space storing the second phase may be arranged between a space storing the first phase and a space storing the third phase. When stored as described, immediately after discharging the cosmetic composition, the second phase may be placed between the first phase and the third phase, so that the composition may be further uniformly mixed.

In an embodiment, the cosmetic may be formulated as one or more of a makeup primer, a makeup base, a foundation, a concealer, a pact, a powder pact, a lipstick, a lip gloss, a powder, an eyeliner, an eyebrow pencil, an eye shadow, a blusher, a sunscreen, a lotion, a cream, an essence, a cleansing oil, and a makeup remover.

In the specification, the ‘cosmetic composition’ is a concept of including all substances that beautify the appearance of a human body. For example, the cosmetic composition may be a cosmetic composition for skin care, a cosmetic composition for bathing, a cosmetic composition for hair, or a cosmetic composition for makeup and specifically, a skin toner, an essence, an emollient cream, a cleansing oil, a makeup remover, an emulsified bodywash, a shampoo, a hair treatment, a hair conditioner, a pore primer, a makeup primer, a makeup base, a foundation, a skin cover, a lipstick, a lip gloss, a face powder, a cheek color, a compact powder, a twin cake, a pack, a powder pact, an eyebrow, an eye shadow, a concealer, a blusher, an airless, and the lie. The composition may further include various functional ingredients and skin nutritional ingredients known in the same technical field, as long as they may not impair the effect. For example, it may further include moisturizers, preservatives, solubilizers, excipients, thickeners, surfactants, suspending agents, antioxidants, pH adjusters, stabilizers, softeners, emulsifiers, colorants, flavoring agents, or combinations thereof. In addition, it may further include an adjuvant commonly used in the field of cosmetology or dermatology such as fatty substances, organic solvents, solubilizers, thickeners, gelling agents, antioxidants, forming agents, fragrances, fillers, sequestering and chelating agents, vitamins, blocking agents, humectants, essential oils, dyes, pigments, hydrophilic or lipophilic active agents, lipid vesicles, or any other ingredients commonly used in cosmetics. And, the above components may be introduced in amounts commonly used in the field of dermatology.

Hereinafter, the present disclosure is illustrated in more detail with reference to examples. However, these examples are exemplary, and the present disclosure is not limited thereto.

### Example 1

### Preparation of First Phase

A first phase is prepared by mixing a thickener including a combination of glycerin, polyacrylate crosspolymer-6, sodium polyacryloyldimethyl taurate, and ammonium acryloyldimethyltaurate/VP copolymer with purified water.

### Preparation of Second Phase

A water gel is prepared by mixing polyol including butylene glycol and glycerin, water, and gellan gum. A primer gel is prepared by mixing a silicone gel (dimethicone*dimethicone crosspolymer), silicone oil (dimethicone, disiloxane), and a film agent (acrylates/polytrimethylsiloxymethacrylate copolymer). The prepared water gel is dispersed in the primer gel in a content shown in Table 1 to prepare a second phase.

### Preparation of Third Phase

An oil-phase component is prepared by mixing volatile oil (a combination of dimethicone and trisiloxane), non-volatile oil (dimethicone), and powder. Subsequently, a pigment and a dispersant/an emulsifier are added thereto and then, dispersed therein and then, emulsified by adding an aqueous phase component of glycerin and butylene glycol mixed in a ratio of 1:1 thereto in a general emulsification method to prepare a third phase. The volatile oil, the non-volatile oil, the powder, the dispersant/the emulsifier, and the aqueous phase component are used in each content shown in ng Table 1.

### Comparative Example 2

A cosmetic composition is prepared in the same manner as in Example 1 except that the second phase is prepared by using silicone gel alone as a component.

### Comparative Example 3

A cosmetic composition is prepared in the same manner as in Example 1 except that the second phase is prepared by using components in contents shown in Table 1 and additionally emulsified with laurylpolyglyceryl-3 polydimethyl siloxyethyldimethicone.

### Comparative Example 4

A cosmetic composition is prepared in the same manner as in Example 1 except that the component contents of the third phase are changed as shown in Table 1.

### Manufacturing of Cosmetics

The prepared first to third phases were injected into each tube of a multi-tube pump container having three tubes to manufacture cosmetics according to Example 1 and Comparative Examples 2 to 4. Cosmetics of Comparative Example 1 was manufactured in the same manner as in Example 1 except that the second phase composition was not used.

TABLE 1

| | | | Comparative | Comparative | Comparative | Comparative |
|---|---|---|---|---|---|---|
| | | Example | Example | Example | Example | Example |
| | | 1 | 1 | 2 | 3 | 4 |
| First phase | | 33 | 33 | 33 | 33 | 33 |
| Second | Silicone gel | 13.5 | — | 20.5 | 13.5 | 13.5 |
| phase | Silicone oil | 11.5 | — | 11.5 | 10.5 | 11.5 |
| | Film agent | 1 | — | 1 | 1 | 1 |
| | Water gel | 7 | — | — | 7 | 7 |
| | Emulsifier | — | — | — | 1 | — |
| Third | Volatile oil | 8 | 8 | 8 | 8 | 10 |
| phase | Non-volatile oil | 2 | 2 | 2 | 2 | — |
| | Dispersant/emulsifier | 2.5 | 2.5 | 2.5 | 2.5 | 2.5 |
| | Powder | 4.5 | 4.5 | 4.5 | 4.5 | 4.5 |
| | Aqueous phase | 17 | 17 | 12 | 17 | 17 |

| | | | | | |
|---|---|---|---|---|---|
| Viscosity of second phase (cps) | 8,000 | — | 30,000 | 6,000 | 8,000 |

### Evaluation

### 1. Sensory Evaluation

A sensory evaluation was conducted to compare feeling of use, when the first to third phase compositions according to an embodiment were simultaneously mixed and when the first to third phase compositions are sequentially used as in conventional makeup steps.

The cosmetic composition according to the example was dispensed from a multi tube pump container containing them on the back of evaluators' hands and then, mixed. The feeling of use was evaluated immediately after the mixing, 8 hours after the mixing, and when additionally using a regular foundation after the mixing. In addition, the first to third phase compositions were respectively in sequence applied to the back of the hand and also, evaluated with respect to the feeling of the use immediately after the mixing, 8 hours after the mixing, and when additionally using a regular foundation after the mixing. This sensory evaluation was conducted by using a general questionnaire with 30 men and women who participated in the evaluation. Table 2 shows the detailed effect items used in the sensory evaluation and satisfaction results according thereto.

TABLE 2

| | | | Use |
|---|---|---|---|
| | | | each |
| | | Simultaneous | in |
| | Detailed effects | mixed use | order |
| Immediately | My skin is moist (not dry). | 93.3% | 83.3% |
| after use | The skin is toned up and there is no | 93.3% | 86.7% |
| | darkening. | | |
| | My skin feels comfortable. | 86.7% | 73.3% |
| | Refreshing and non-sticky. | 86.7% | 73.3% |
| | It's light, not stuffy, and not heavy. | 86.7% | 73.3% |
| | Average satisfaction | 89.34% | 77.98% |
| 8 hours | The comfortable feeling inside the | 83.3% | 76.7% |
| later | skin lasts a long time even after time | | |
| | passes. | | |
| | Even as time passes, pores are well | 66.7% | 63.3% |
| | covered and skin looks smooth. | | |
| | It looks natural and unpretty even as | 83.3% | 73.3% |
| | time passes. | | |
| | It remains refreshing and non-sticky | 80.0% | 76.7% |
| | even after time passes. | | |
| | Even as time passes, it is light, not | 83.3% | 73.3% |
| | stuffy, and not heavy. | | |
| | Average satisfaction | 79.32% | 72.66% |
| When used | Skin condition looks good. | 86.7% | 73.3% |
| with | It adheres well to foundation. | 76.7% | 66.7% |
| foundation | Refreshing and non-sticky. | 86.7% | 70.0% |
| after mixing | It's light, not stuffy, and not heavy. | 90.0% | 70.0% |
| | It doesn't get stuck in pores easily. | 83.3% | 73.3% |
| | Average satisfaction | 84.68% | 70.66% |

Referring to Table 2, as a result of comparing feeling of use immediately after the mixing, the case of simultaneously mixing three formulations exhibited a high average satisfaction level of 89.34%, while the other case of sequentially using the three chemical formulations exhibited an average satisfaction level of 77.98%. Specifically, the case of simultaneously mixing three chemical formulations was evaluated to exhibit more moist skin, excellent tone-up effect, no darkening, and refreshing and light feeling of use rather than sticky or heavy feeling of use. This is understood to result from uniform mixing of the three chemical formulations.

As a result of comparing feeling of use of the two usage modes 8 hours after the mixing, the case of simultaneously using three chemical formulations exhibited an average satisfaction level of 79.32%, compared with the other case of sequentially using three chemical formulations exhibited an average satisfaction level of 72.66%. Specifically, the case of simultaneously using three chemical formulations was evaluated to achieve a more natural skin texture and provide not heavy but fresh feeling of use.

As a result of comparing the two usage modes, when the regular foundation was additionally used after the mixing, the case of simultaneously using three chemical formulations exhibited an average satisfaction level of 84.68%, but the other case of sequentially using three chemical formulations exhibited a much high average satisfaction level of 70.66%. Specifically, in the case of simultaneously using three chemical formulations, the foundation used after the was evaluated to have better adhesion thereto, not get stuck in pores, and provide fresh and light feeling of use rather than sticky or heavy feeling of use.

### 2. Mixed Evaluation

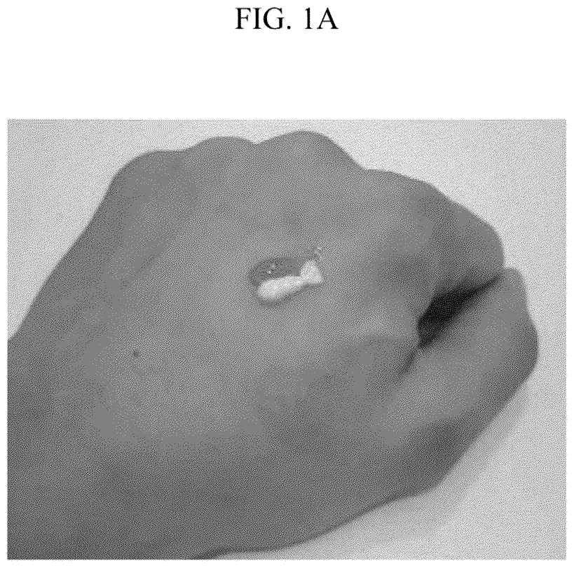

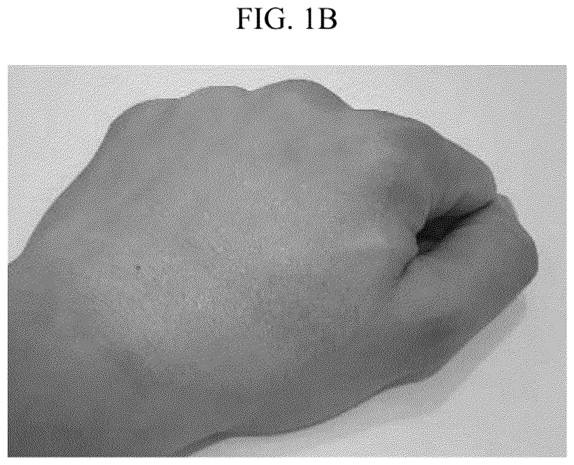

The compositions according to the example and the comparative examples were respectively dispensed from a container onto the back of the hand and then, mixed. The mixing evaluation was performed by observing their appearance immediately after discharging each composition, during the mixing, and after the mixing, and the results are as shown in FIGS. 1A, 1B, 2A, 2B, and 3 to 5.

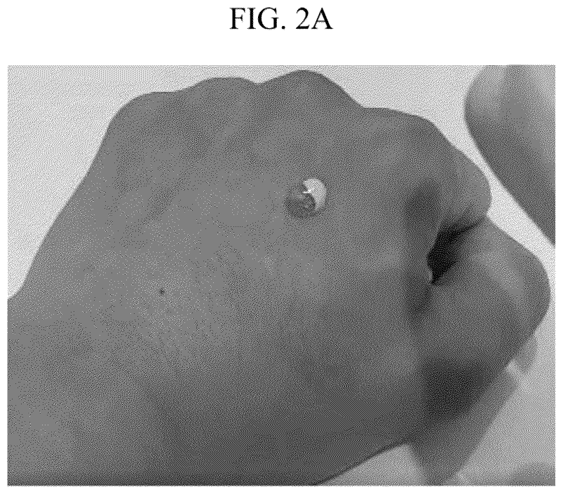

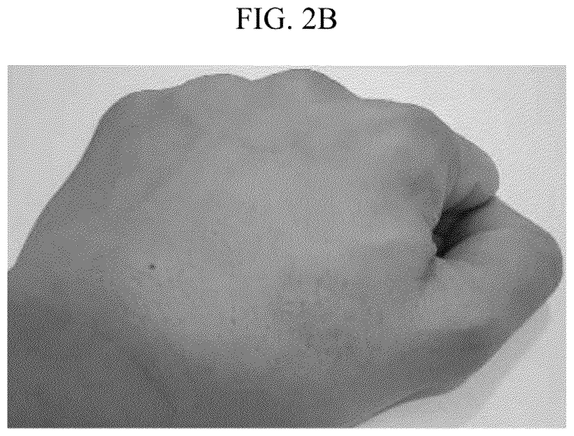

FIG. 1A is a photograph showing the appearance of the composition according to Comparative Example 1 immediately after dispensed from the container, which confirmed that two chemical formulations were dispensed. FIG. 2A is a photograph showing the appearance of the composition according to the example immediately after dispensed from the container, which confirmed that three chemical formulations were discharged. As shown in the appearance immediately after dispensed in FIG. 2A, the multi-pipe pump container had three pipes arranged side by side, which corresponded to the first phase, the second phase, and the third phase in order from the left.

FIG. 1B shows a mixed appearance of the composition of Comparative Example 1 including two chemical formulations, wherein the two chemical formulations were relatively difficult to mix, and the composition was not absorbed but float white on the skin. FIG. 2B shows a mixed appearance of the composition of Example 1 including three chemical formulations, which were uniformly mixed, and after the mixing, the composition was observed to be naturally adhered to the skin.

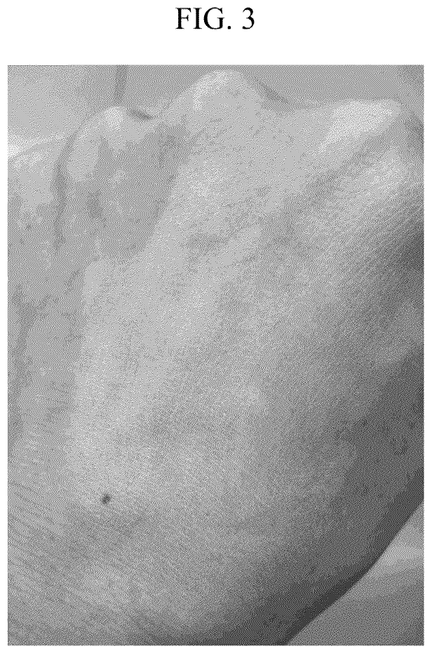

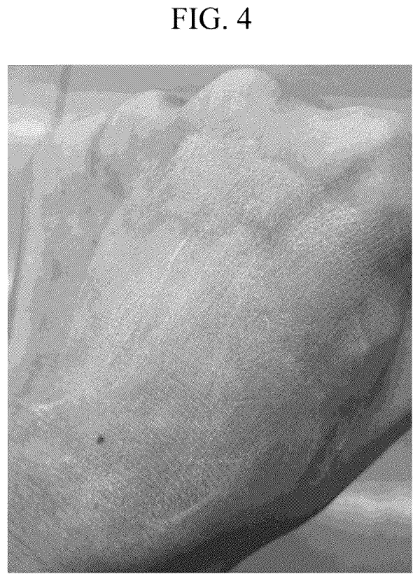

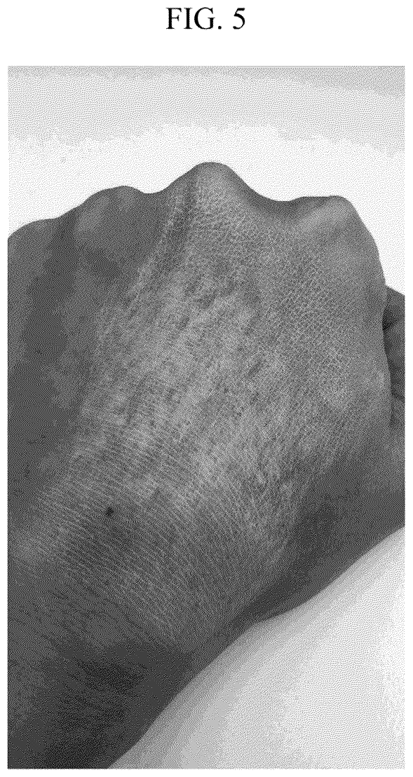

FIG. 3 is a photograph showing the appearance during mixing of a composition according to Example, FIG. 4 is a photograph showing the appearance during mixing of the composition according to Comparative Example 2, and FIG. 5 is a photograph showing the appearance during mixing of the composition according to Comparative Example 3.

Referring to FIG. 4, the composition of Comparative Example 2 using silicone gel alone as the second phase, compared with the composition of the example, was not only uniformly mixed, but also even after the mixing, the composition was not absorbed but rolled around, resulting in unpleasant feeling of use.

As for the composition of Comparative Example 3 using the second phase but by emulsifying it, the second phase was dispensed in a smaller amount than that of the composition of the example, when dispensed from the container, and in addition, when mixed, the composition of Comparative Example 3 exhibited no uniform mixing of the first to third phases, compared with that of the example.

Referring to FIG. 5, the composition of Comparative Example 4 using the third phase including a large amount of volatile oil was not mixed, because the pigment was first set on the skin but remained white on the skin, when mixed.

Although the preferred embodiments of the present invention have been described in detail, the scope of the present invention is not limited thereto, and various modifications and improvements by those skilled in the art using the basic concept of the present invention defined in the following claims are also within the scope of the invention.

What is claimed is:

1. A cosmetic composition including three different chemical formulations, wherein the first phase includes an oil-in-water (O/W) type or an aqueous dispersion, the second phase includes a silicone gel and a water gel, and the third phase includes a water-in-silicone (W/S) type or a water-in-oil (W/O) type.

2. The cosmetic composition of claim 1, wherein the first phase includes polyacrylate cross polymer-6, sodium polyacryloyldimethyl taurate, ammonium acryloyldimethyltaurate/VP copolymer, a polyol, purified water, or a combination thereof.

3. The cosmetic composition of claim 1, wherein the second phase includes silicone gel in an amount of 35 wt % to 90 wt % based on a total weight of the second phase.

4. The cosmetic composition of claim 1, wherein the second phase is a silicone-in-water (W/S) type including an oil-phase component, and the oil-phase component is included in an amount of 60 wt % to 90 wt % based on a total weight of the second phase.

5. The cosmetic composition of claim 1, wherein the water gel includes gellan gum, xanthan gum, galactomannan, glucomannan, guar gum, locust bean gum, pluronic, agar, carrageenan, or a combination thereof.

6. The cosmetic composition of claim 1, wherein the third phase includes volatile oil selected from isooctane, isodecane, isododecane, isohexadecane, isoparaffin, dimethylpolysiloxane, cyclomethicone, cyclotetrasiloxane, cyclopentasiloxanecyclohexasiloxane, phenyl trimethicone, methyl trimethicone, dimethicone, trisiloxane, or a combination thereof.

7. The cosmetic composition of claim 1, wherein the third phase includes volatile oil, and the volatile oil is included in an amount of 25 wt % or less based on a total weight of the cosmetic composition.

8. The cosmetic composition of claim 1, wherein the cosmetic composition includes 10 to 60 wt % of the first phase, 10 to 60 wt % of the second phase, and 10 to 60 wt % of the third phase based on a total weight of the composition.

9. The cosmetic composition of claim 1, wherein the cosmetic composition includes 100 to 150 parts by weight of the first phase and the third phase, respectively, based on 100 parts by weight of the second phase.

10. The cosmetic composition of claim 1, wherein viscosities of the first to third phases are each 15,000 cps or less.

11. The cosmetic composition of claim 1, wherein the first phase, the second phase, and the third phase in the cosmetic composition are not mixed with each other, but are mixed immediately before use.

12. The cosmetic composition of claim 11, wherein the cosmetic composition is such that the second phase is located between the first phase and the third phase prior to use.

13. A cosmetic including the cosmetic composition of claim 1 in a multi-tube container.

14. The cosmetic of claim 13, wherein the cosmetic is used as one or more of a makeup primer, a makeup base, a foundation, a concealer, a pact, a powder pact, a lipstick, a lip gloss, a powder, an eyeliner, an eyebrow pencil, an eye shadow, a blusher, a sunscreen, a lotion, a cream, an essence, a cleansing oil, and a makeup remover.
